(19) United States
(12) Patent Application Publication
(10) Pub. No.: US 2025/0002457 A1
(43) Pub. Date: Jan. 2, 2025

(54) N,N-DIMETHYLTRYPTAMINE (DMT) CRYSTALLINE PRODUCTS AND METHODS OF MAKING THE SAME

(71) Applicant: Psilera Inc., Tampa, FL (US)

(72) Inventors: Jeanine Yacoub, Lakeland, FL (US); Jacqueline Salm, Tampa, FL (US); Christopher Witwoski, Tampe, AL (US)

(21) Appl. No.: 18/689,889

(22) PCT Filed: Oct. 24, 2022
(86) PCT No.: PCT/US2022/047520

§ 371 Date: Mar. 7, 2024

Related U.S. Application Data
(60) Provisional application No. 63/273,673, filed on Oct. 29, 2021.

(51) Int. Cl. C07D 209/16

(52) U.S. Cl. CPC C07D 209/16

(57) ABSTRACT

An effective and efficient process for obtaining a crystalline products comprised of salts of N,N-Dimethyltryptamine (DMT) freebase is provided. The crystalline products may, for example, be DMT fumarate, DMT tartrate, DMT succinate, or DMT maleate.

### CROSS REFERENCE TO RELATED APPLICATION

This application is a national stage application of PCT/US2022/047520, filed Oct. 24, 2022, which claims the benefit of both the filing date of claims the benefit of the filing date of U.S. provisional patent application Ser. No. 63/273,673, filed Oct. 29, 2021, the entire disclosure of which is incorporated by reference.

### FIELD OF THE INVENTION

The present invention relates to the field of crystalline forms of N,N-Dimethyltryptamine (DMT).

### BACKGROUND OF THE INVENTION

Psychoactive drugs are compounds that affect behavior, mood, thought, or perception. Psychoactive drugs include antipsychotics, anti-anxiety agents, stimulants, reuptake inhibitors, monoamine oxidase inhibitors (MAOI), tricyclic antidepressants, and mood stabilizers. Some of these compounds have historically been used for off-label psychoactive activity and are now being investigated for positive clinical efficacy.

Indole compounds represent a diverse class of compounds with broad biomedical potential across many targets including cancer, cardiovascular, gastrointestinal, and a wide range of neurological disorders, including ones for which known psychoactive drugs have been used. N,N-dimethyltryptamine (DMT) is a naturally occurring psychedelic indole compound that has recently become of particular interest for therapeutic applications. In addition to being available in nature, DMT can be synthesized by reductive amination of tryptamine.

However, many known processes for obtaining DMT from naturally occurring sources, as well as synthesizing it and its salt form, are unduly cumbersome. Thus, there is a need for new methods for creating DMT and salts thereof.

### SUMMARY OF THE INVENTION

The present invention is directed to new crystalline forms (also referred to as crystalline products) of DMT, methods for making these new crystalline forms, and methods of using these new crystalline forms. The crystalline products are formed by the crystallization or recrystallization of DMT from DMT freebase that has been exposed to an acid salt, thereby generating salts DMT such as pharmaceutically acceptable salts of DMT.

According to a first embodiment, the present invention provides a method of producing an N,N-dimethyltryptamine (DMT) crystalline product. This method comprises: (a) dissolving DMT freebase in a solvent; (b) adding an acid to form a slurry; (c) filtering the slurry to generate a residue; and (d) forming a crystalline product from the residue, wherein the crystalline product comprises DMT and a conjugate base of the acid.

According to a second embodiment, the present invention provides crystalline N,N-dimethyltryptamine (DMT) fumarate, wherein the DMT fumarate forms a unit cell in which there is a ratio of DMT:fumarate of about 3:1 to 1.5:1.

According to a third embodiment, the present invention provides crystalline N,N-dimethyltryptamine (DMT) tartrate, wherein the DMT tartrate forms a unit cell in which there is a ratio of DMT:tartrate of about 0.5:1 to 1.5:1.

According to a fourth embodiment, the present invention provides crystalline N,N-dimethyltryptamine (DMT) succinate, wherein the DMT succinate forms a unit cell in which there is a ratio of DMT:succinate of about 3.0:1 to 0.5:1.

According to a fifth embodiment, the present invention provides crystalline N,N-dimethyltryptamine (DMT) maleate, wherein the DMT maleate forms a unit cell in which there is a ratio of DMT:maleate of about 3.0:1 to 0.5:1.

According to a sixth embodiment, the present invention provides crystalline N,N-dimethyltryptamine (DMT) fumarate, wherein the DMT fumarate is characterized by at least one of: (a) unit cell dimensions of a=7.7447(3) Å, b=9.3258(4) Å, c=12.4691(4) Å, α=102.798(2) Å, β=104.869(2) Å, γ=103.270(2) Å, at a temperature of about 298° K; (b) a triclinic crystal system and a P-1 space group at a temperature of about 298° K; and (c) an x-ray powder diffraction pattern with peaks at 20.5 and 25.0° 2θ±0.2° 2θ.

According to a seventh embodiment, the present invention provides crystalline N,N-dimethyltryptamine (DMT) tartrate, wherein the DMT tartrate is characterized by one or both of one of: (a) unit cell dimensions of a=7.57270(10) Å, b=9.52180(10) Å, c=23.7834(4) Å, α=90°, β=90.6530(10)°, and γ=90°, at a temperature of about 297° K; and a monoclinic crystal system and a P2₁/n space group at a temperature of about 297° K.

According to an eighth embodiment, the present invention provides methods of preventing or treating a physical and/or psychological and/or psychiatric condition and/or other neurologic condition. The method comprises administration of an effective amount of a crystalline form of DMT of the present invention to a subject in need. In some embodiments, the crystalline form may be produced according to a method of the present invention.

According to a ninth embodiment, the present invention is directed to the use of an effective amount of a crystalline form of DMT of the present invention to prevent or treat a physical and/or psychological and/or psychiatric condition and/or other neurologic condition.

According to a tenth embodiment, the present invention is directed to a medicament comprising an effective amount of crystalline form of DMT of the present invention and/or a crystalline form of DMT made according to a method of the present invention for preventing or treating a physical and/or psychological and/or psychiatric condition and/or other neurologic condition.

According to an eleventh embodiment, the present invention is directed to a pharmaceutical product comprising a crystalline product of the present invention and an excipient.

Through the various embodiments of the present invention, one can effectively and efficiently create DMT crystalline products. Among the advantages of various embodiments of the present invention are one or both of a shorter synthetic pathway than is necessary in traditional Speeter Anthony procedures for synthesizing DMT and the ability to forego the use of column chromatography and other cumbersome purification processes. These products may be used in existing and new formulations to prevent and/or treat physical and/or psychological and/or psychiatric conditions and/or other neurologic conditions.

### BRIEF DESCRIPTION OF THE FIGURES

FIG. 1 is a representation of an ellipsoid plot of DMT fumarate.

FIG. 2 shows the crystal packing of DMT fumarate along the b axis.

FIG. 3 shows the crystal packing of DMT fumarate illustrating a hydrogen bonding layer

FIG. 4 is a simulated X-ray powder diffraction pattern for DMT fumarate as compared to an x-ray powder diffraction from a prior art compound with a Y-Offset and the same prior art material with both a Y-Offset and an X-Offset.

FIG. 5 is a representation of an ellipsoid plot of DMT tartrate.

FIG. 6 is a representation of the hydrogen bonding layer of DMT tartrate in which, for illustration purposes, the ratio of cations is not preserved.

FIG. 7 is a representation of packing and hydrogen bonding in a DMT tartrate structure.

### DETAILED DESCRIPTION OF THE INVENTION

Reference will now be made in detail to various embodiments of the present invention, examples of which are illustrated in the accompanying figures. In the following description, numerous specific details are set forth in order to provide a thorough understanding of the present invention. However, unless otherwise indicated or implicit from context, the details are intended to be examples and should not be deemed to limit the scope of the invention in any way. Additionally, features described in connection with the various or specific embodiments are not to be construed as not appropriate for use in connection with other embodiments disclosed herein unless such exclusivity is explicitly stated or implicit from context.

Headers are provided herein for the convenience of the reader and do not limit the scope of any of the embodiments disclosed herein.

### Definitions

Unless otherwise stated or implicit from context the following terms and phrases have the meanings provided below.

The indefinite articles “a” and “an” and the definite article “the” include plural as well as singular referents, unless the context clearly dictates otherwise.

The terms “about” and “approximately” means an acceptable error for a particular value as determined by one of ordinary skill in the art, which depends in part on how the value is measured or determined. In certain embodiments, the term “about” or approximately” means within 1, 2, 3 or 4 standard deviations. In certain embodiments, the term “about” or “approximately” means with 30%, 20%, 15%, 10%, 9%, 8%, 7%, 6%, 5%, 4%, 3%, 2%, 1%, 0.5%, 0.1%, or 0.05%, of a given value or range.

The phrase “abuse disorder” refers to a disorder or disease that affects a person's brain and behavior and leads to an inability to control the use of a legal or illegal drug or medication. Prescription medicines, non-prescription medicines, and non-approved drugs may all be abused drugs, the use of which may lead to an abuse disorder. Drugs and medications may also include substances such as amphetamines, opioids, cocaine, barbiturates, alcohol, marijuana, and nicotine.

A “crystalline product” is a product in which molecules are arranged in an

ordered state as opposed to an amorphous state. Crystalline product may include one type of molecule or a plurality of types of molecules. A crystalline product may also be referred to as a crystalline form.

“DMT” refers N,N-dimethyltryptamine, which is a molecule that has the following structure:

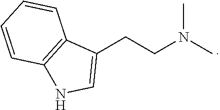

“DMT salt” refers to the combination of DMT with a conjugate base in which the DMT has the following structure:

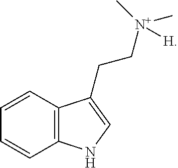

By way of example, when the conjugate base is the conjugate based of fumaric acid, it has the following structure:

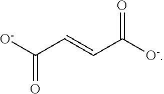

A DMT salt may be a pharmaceutically acceptable salt.

The terms “manage,” “managing,” and “management” refer to preventing or slowing the progression, spread or worsening of a disease or disorder, or of one or more symptoms thereof. Often, the beneficial effects that a subject derives from a prophylactic and/or therapeutic agent do not result in a cure of the disease or disorder. In this regard, the term “managing” encompasses treating a subject who has suffered from the particular disease in an attempt to prevent or minimize the recurrence of the disease.

A “mood disorder” refers to a group of conditions in which a disturbance in the person's mood is the underlying feature. Mood disorders may be groups of mania (elevated mood disorders) or hypomania (depression). The classification is in the Diagnostic and Statistical Manual of Mental Disorders (DSM) and the International Classification of Diseases (ICD).

A “neurological disorder” refers to diseases of the central and peripheral nervous system e.g., the brain, spinal cord, cranial nerves, peripheral nerves, nerve roots, autonomic nervous system, neuromuscular junction, and muscles. These disorders include epilepsy, Alzheimer's disease and other dementias, cerebrovascular diseases including stroke, migraine, cluster headaches and other headache disorders, multiple sclerosis, Parkinson's disease, neuro-infections, brain tumors, traumatic disorders of the nervous system due to head trauma, and traumatic disorders due to traumatic or terrifying experiences (Posttraumatic Stress Disorder e.g., PTSD) and neurological disorders as a result of malnutrition and substance abuse. The substance abused may be any number of addictive substances, especially alcohol and drugs and combinations thereof. Additionally, many bacterial (e.g., Mycobacterial tuberculosis, Neisseria meningitides), viral (e.g., Human Immunodeficiency Virus (HIV), Lyme Disease, Enteroviruses, West Nile Virus, Zika), fungal (e.g., Cryptococcus, Aspergillus), and parasitic (e.g., malaria, Chagas) infections can affect the nervous system and lead to neurological disorders. Neurological symptoms that accompany these disorders may occur because of an infection itself, and/or an immune response.

A “pharmaceutically acceptable salt” is a salt that is of sufficient purity and quality for use in a formulation of a composition or medicament of the present invention. Both human use (clinical and over-the-counter) and veterinary use are included within the scope of the present invention. A formulation of the present invention includes a composition or medicament for either human or veterinary use. Pharmaceutically acceptable salts, include but are not limited to acid addition salts that have been formed with the free amino groups of a protein.

The terms “prevent,” “preventing,” and “prevention” refer to the prevention of the onset, recurrence or spread of a disease or disorder or of one or more symptoms thereof. In certain embodiments, the terms refer to the treatment with or administration of a compounder dosage, with or without one or more other additional active agent(s), prior to the onset of symptoms, particularly to a subject at risk of diseases or disorders provided herein. The terms encompass the inhibition or reduction of a symptom of the particular disease. Subjects with familial history of a disease in particular are candidates for preventive regimes in certain embodiments. In addition, subjects who have a history of recurring symptoms are also potential candidates for prevention. In this regard, the term “prevention” may be interchangeably used with the term “prophylactic treatment.”

A “prophylactically effective amount” of a compound is an amount sufficient to prevent a disease or disorder or prevent its recurrence. A prophylactically effective amount of a compound means an amount of therapeutic agent, alone or in combination with one or more other agent(s), that provides a prophylactic benefit in the prevention of the disease. The term “prophylactically effective amount” can encompass an amount that improves overall prophylaxis or enhances the prophylactic efficacy of another prophylactic agent.

The term “subject” is defined herein to include animals such as mammals, including, but not limited to, primates (e.g., humans), cows, sheep, goats, horses, dogs, cats, rabbits, rats, mice, and the like. In specific embodiments, the subject is a human.

As used herein, and unless otherwise specified, the terms “therapeutically effective amount” and “effective amount” mean an amount sufficient to provide a therapeutic benefit in the treatment or management of a disease or disorder, or to delay or minimize one or more symptoms associated with the disease or disorder. The terms “therapeutically effective amount” and “effective amount” can encompass an amount that improves overall therapy, reduces or avoids symptoms or causes of disease or disorder, or enhances the therapeutic efficacy of another therapeutic agent.

The terms “treat,” “treating,” and “treatment” refer to the eradication or amelioration of a disease or disorder, or of one or more symptoms associated with the disease or disorder. In certain embodiments, the terms refer to minimizing the spread or worsening of the disease or disorder by administering of one or more prophylactic or therapeutic agents to a subject with such disease or disorder. In some embodiments the terms refer to the administration of a compound or dosage form provided herein, with or without one or more additional active agent(s), after the onset of symptoms of a particular disease.

### Crystalline Products

Various embodiments of the present invention are directed to crystalline forms of DMT. In some embodiments, the crystalline products are salts of DMT, such as DMT fumarate, DMT maleate, DMT succinate, and DMT tartrate. Thus, in some embodiments, the compositions of the present invention comprise, consist essentially of, or consist of DMT and fumarate; DMT and maleate; DMT and succinate; or DMT and tartrate.

As persons of ordinary skill in the art are aware, crystals may be characterized in a number of different ways. In some embodiments, the product is DMT fumarate in which the DMT fumarate forms a unit cell in which there is a ratio of DMT:fumarate of about 3:1 to 1.5:1 or about 2:1.

Certain crystalline N,N-dimethyltryptamine (DMT) fumarate products of the present invention may be characterized by at least one of, at least two of or all three of: unit cell dimensions of a=7.7447(3) Å, b=9.3258(4) Å, c=12.4691(4) Å, α=102.798(2)°, β=104.869(2)°, and γ=103.270(2)°, at a temperature of about 298° K; (b) a triclinic crystal system and a P-1 space group at a temperature of about 298° K;

and (c) an x-ray powder diffraction pattern with peaks at 20.5 and 25.0° 2θ±0.2° 2θ. In some embodiments, the unit cell volume is about 808.67(5) Å.

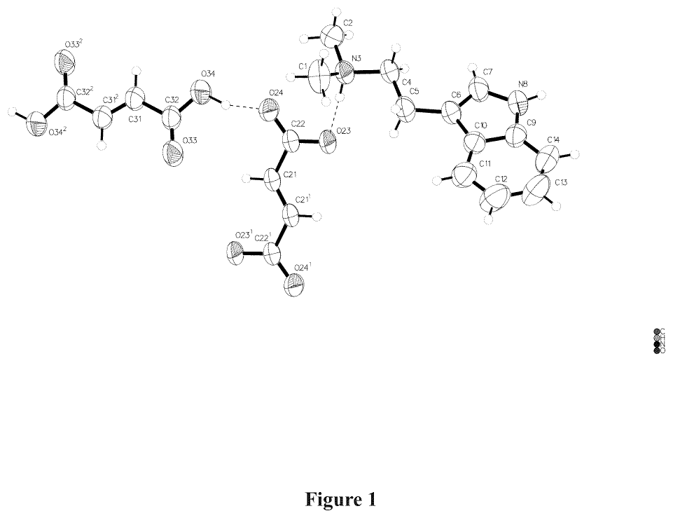

FIG. 1 shows an ellipsoid plot of DMT fumarate. The components may be associated with each other at N3 of the DMT moiety and 023 of the fumarate moiety.

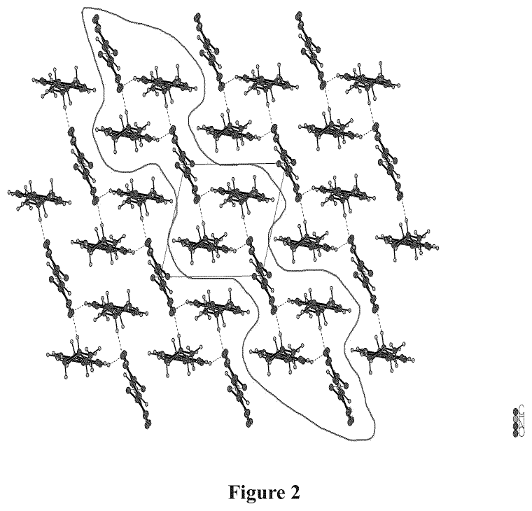

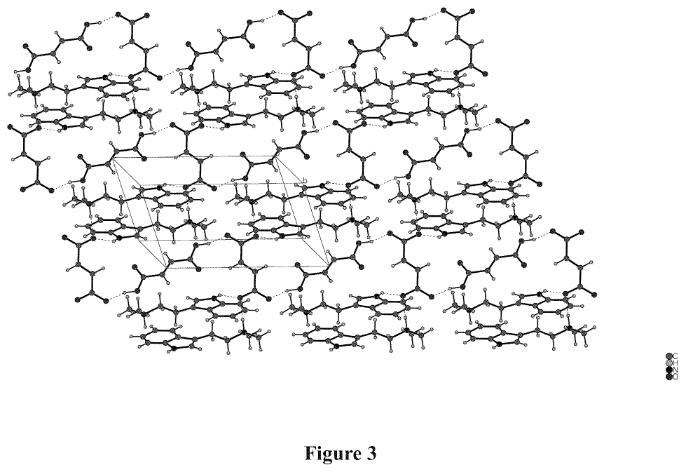

In some embodiments, the crystalline products of the present invention form a structure that has layers that maintain hydrogen bonds relative to one another. The packing of these hydrogen layers in DMT fumarate may be seen as viewed along the b direction in FIG. 2 and in a hydrogen bonding layer as shown in FIG. 3. The crystallization structure is orthorhombic and it is centrosymmetric, i.e., non-chiral.

In some embodiments, the present invention is directed to a DMT tartrate product that forms a unit cell in which there is a ratio of DMT:tartrate of about 0.5:1 to 1.5:1 or about 1:1.

Certain crystalline N,N-dimethyltryptamine (DMT) tartrate crystalline products of the present invention may be characterized by at least one of, at least two of or all three of: unit cell dimensions of a=7.57270(10) Å, b=9.52180(10) Å, c=23.7834(4) Å, α=90°, β=90.6530(10)°, and γ=90°, at a temperature of about 297° K; (b) a monoclinic crystal system and a P2₁/n space group at a temperature of about 297° K. In some embodiments, the unit cell volume is about 1714.81(4) Å. Alternatively or additionally, the product may have the following characteristics a=7.5569(1) Å, b=9.4024(2) Å, c=23.7570(4) Å, α=90°, β=90.742(1)°, and γ=90° at a temperature of about 100° K.

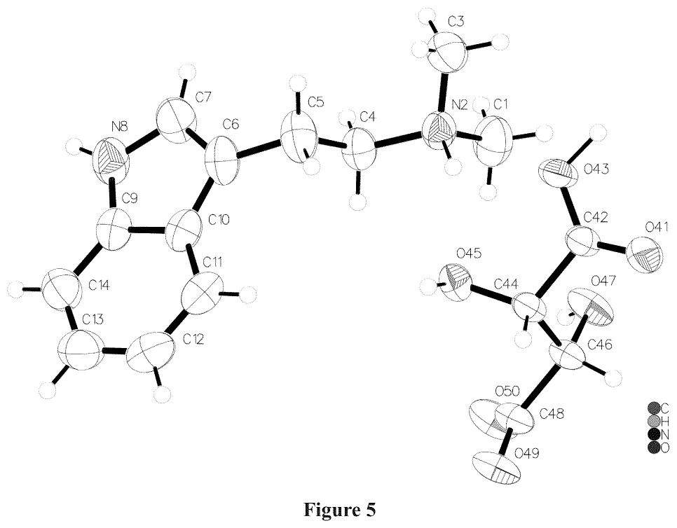

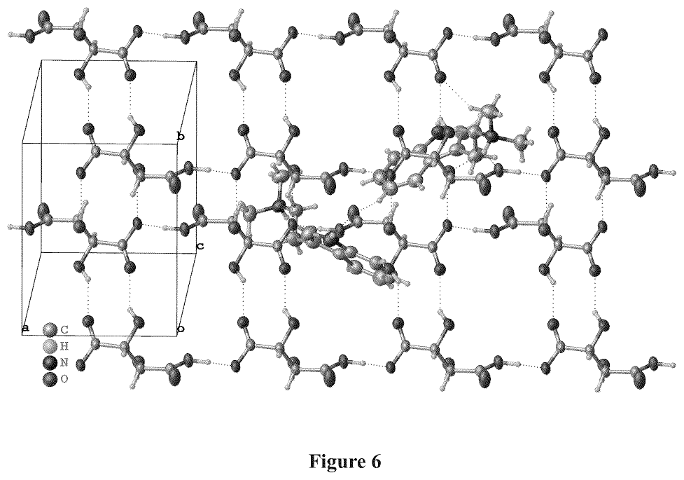

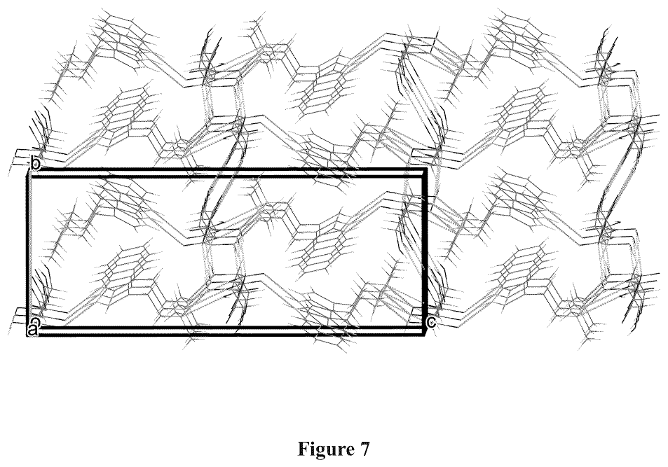

FIG. 5 is an ellipsoid plot that shows that in the DMT tartrate, the DMT cation ion is disordered. The crystal that is formed is a salt, and as shown in FIG. 6, singly deprotonated tartrate anions form an OH . . . O singly bonded hydrogen layer. As shown in FIG. 7, layers are connected through N—H . . . O hydrogen bonds with DMT cations. Thus, strong, charge assisted hydrogen bonds are present in the tartrate hydrogen bonded layer.

### Generation of DMT Freebase

The present invention is not limited to any particular method for generating the DMT freebase. However, an example of how to generate the DMT freebase uses reductive amination of tryptamine. A freebase is the conjugate base (deprotonated) form of an amine, and is often referred to as such with alkaloids and similar amine-containing compounds.

One may dissolve tryptamine in an organic acid and alcohol. Examples of organic acids that may be used to dissolve tryptamine include but are not limited to acetic acid, formic acid, or similar organic acid. Examples of alcohols that may be used to dissolve tryptamine include but are not limited to methanol, ethanol, isopropyl, or similar organic alcohol solvents.

Next one adds a reducing agent such as sodium cyanoborohydride or sodium triacetoxyborohydride. Subsequently, one adds a formaldehyde solution, e.g., formaldehyde in methanol, under conditions that allow for the creation of DMT. After the DMT has been synthesized, the mixture may be concentrated under a vacuum. The concentrated material forms a residue that may be dissolved in an organic substance such as dichloromethane and a base such as NaOH. Next one may dry the material on an agent such as sodium sulfate and subject it to a vacuum for concentration to yield a solid material. This solid material is a crude mixture that contains DMT freebase and a nitrile by product at one of the N-methyl substituents (N—CH₂CN) and that can be used in the crystallization processes describe below.

### Crystallization Methods

In some embodiments, a crystallization methodology is used to generate a crystalline product. This process may, for example, begin with a composition that comprises, consists essentially of or consists of DMT freebase that may or may not be generated according to the method described above. The DMT freebase may be dissolved in a solvent such as an organic solvent, e.g., chloroform, acetone, and isopropyl alcohol.

The dissolved DMT freebase may be combined with an acidic solution to form a slurry. The acidic solution may, for example, comprise, consist essentially of or consist of an organic acid and a solvent. Examples of organic acids that made be used include, but are not limited to, fumaric aid, tartaric acid, succinic acid, benzoic acid, maleic acid, p-toluenesulfonic acid, hydrochloric acid, hydrobromic acid, acetic acid, sulfuric acid, and phosphoric acid. Examples of solvents for the organic acids include, but are not limited to, acetone isopropyl alcohol, methanol, ethanol, and chloroform. In some embodiments, the acidic solution is heated to a temperature of about 40° C. to 80° C. or 50° C. to 70° C. prior to combination with the DMT freebase that has been dissolved in a solvent. In other embodiments, the acidic solution is at room temperature.

One may next filter the slurry. The DMT crystalline product, may be crystallized by employing a solvent, for example, an alcohol, such as isopropyl alcohol, thereby generating an acid salt of DMT (i.e., the DMT freebase and its conjugate base). When the organic acid is fumaric acid, then the DMT crystalline product will be DMT fumarate. When the organic acid is tartaric acid, then the DMT crystalline product will be DMT tartrate. When the organic acid is succinic acid, then the DMT crystalline product will be DMT succinate. When the organic acid is maleic acid, then the DMT crystalline product will be DMT maleate.

### Methods of Use

The products of the present invention and the methods for creating DMT salts of the present invention may be used to form or be part of medicaments, formulations, or other compositions for treating, preventing, or ameliorating a disease, disorder or condition. Examples of diseases, disorders and conditions, include but are not limited to mood disorders, neurological disorders, and abuse disorders.

These products may be given prophylactically or therapeutically to subjects in need there of and be given in prophylactically effective amounts or therapeutically effective amounts. Methods for administering these compositions include but are not limited to orally, intranasally, through a transdermal route, intravenously, or through any other route that a person of ordinary skill in the art would deem effective for delivery of a DMT salt to a subject.

### EXAMPLES

### Example 1: Generation of DMT Freebase

Tryptamine (3 g, 1 eq.) was dissolved in acetic acid (5.35 mL, 5 eq.) and methanol (120 mL) before addition of sodium cyanoborohydride (2.47 g, 2.1 eq.). The reaction mixture was kept at 0° C. Formaldehyde (39% in methanol, 3.49 mL, 2.6 eq.) was added dropwise and allowed to mix at room temperature until reaction was complete.

The reaction mixture was concentrated under a vacuum and the resulting residue was dissolved in dichloromethane. 1.0 M sodium hydroxide was added. This procedure was done in triplicate, combining organic phases, drying on sodium sulfate, and concentration under vacuum to produce an off-white solid (0.51 g, 45% yield).

### Example 2: Generation DMT Fumarate

The DMT freebase crude mixture from above (example 1) was dissolved in chloroform (125 mL) and added to a hot solution of fumaric acid (1.09 g) in acetone (31 mL). The resulting slurry was filtered and DMT fumarate (1.85 g) was recrystallized from isopropyl alcohol to yield white needles.

Purity was established using liquid chromatography-mass spectrometry to show DMT fumarate m/z=327 (DMT fumarate+Na⁺) at 0.49 min retention time and liberated DMT freebase m/z=189 (DMT+H⁺) at 1.50 min. Peak areas show the purity of DMT to be >98% by area relative to any impurities (UV=254 nm).

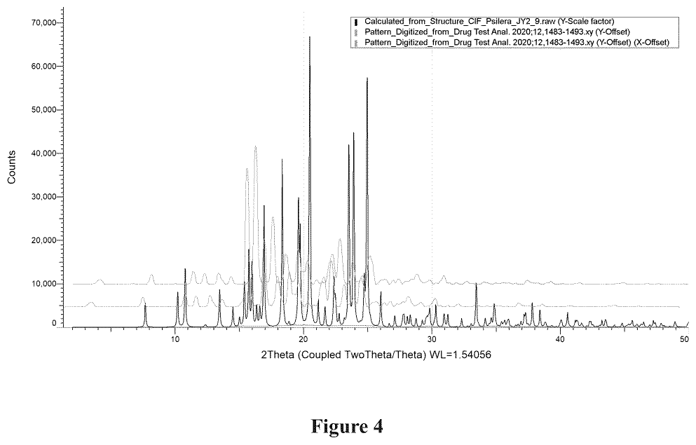

Crystallography was also performed to show unit cell (DMT:fumarate, 2:1) and X-ray diffraction that differs from literature. The differences are shown in FIG. 4, which compares: (1) DMT fumarate of the present invention, the lowest X-ray diffraction plot is that of a DMT fumarate composition of the present invention, to (2) a prior art composition, the upper X-ray diffraction plot is from Drug Test Anal 2020; 12, 1483-1493.xy with a Y-offset, and the middle X-ray diffraction plot is from Drug Test Anal 2020; 12, 1483-1493.xy with a Y-offset and an X-Offset in order to match some of the peaks in the calculated pattern of the product of the present invention.

The differences in plots and their peaks, demonstrate different crystalline structures. The DMT free base mixture (approximately 30 mg) from above is dissolved in acetone (approximately 2-3 mL). Separately, tartaric acid (approximately 25 mg) is dissolved in isopropyl alcohol (approximately 2-3 mL) and heated while stirring to 70-80° C. The hot tartaric acid alcohol solution is added to the room temperature (RT) DMT acetone solution. This mixture is dried down to supersaturate the solution (approximately 1-2 mL), then more RT acetone is added to precipitate out DMT tartrate salt crystals. This is rinsed thoroughly with acetone to remove any impurities, and the supernatant is removed prior to drying the solid crystals under nitrogen.

### Example 3: Generation DMT Tartrate

The DMT free base mixture (approximately 30 mg) from above (example 1) was dissolved in acetone (approximately 2-3 mL). Separately, tartaric acid (approximately 25 mg) was dissolved in isopropyl alcohol (approximately 2-3 mL) and heated while stirring to 70-80° C. The hot tartaric acid alcohol solution was added to the room temperature (RT) DMT acetone solution. This mixture was dried down to supersaturate the solution (approximately 1-2 mL), then more RT acetone was added to precipitate out DMT tartrate salt crystals. This was rinsed thoroughly with acetone to remove any impurities, and the supernatant was removed prior to drying the solid crystals under nitrogen.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. Crystalline N,N-dimethyltryptamine (DMT) fumarate, wherein the DMT fumarate is characterized by at least one of:
(A) unit cell dimensions of
a=7.7447(3) Å,
b=9.3258(4) Å,
c=12.4691(4) Å,
α=102.798(2)°,
β=104.869(2)°, and
γ=103.270(2)°,
at a temperature of about 298° K;
(B) a triclinic crystal system and a P-1 space group at a temperature of about 298° K; and
(C) an x-ray powder diffraction pattern with peaks at 20.5 and 25.0° 2θ±0.2° 2θ.

20. The crystalline N,N-dimethyltryptamine (DMT) fumarate of claim 19, wherein the DMT fumarate is characterized by at least two of:
(A) unit cell dimensions of
a=7.7447(3) Å,
b=9.3258(4) Å,
c=12.4691(4) Å,
α=102.798(2)°,
β=104.869(2)°, and
γ=103.270(2)°,
at a temperature of about 298° K;
(B) a triclinic crystal system and a P-1 space group at a temperature of about 298° K; and
(C) an x-ray powder diffraction pattern with peaks at 20.5 and 25.0° 2θ±0.2° 2θ.

21. The crystalline N,N-dimethyltryptamine (DMT) fumarate of claim 20, wherein the DMT fumarate is characterized by all three of:
(A) unit cell dimensions of
a=7.7447(3) Å,
b=9.3258(4) Å,
c=12.4691(4) Å,
α=102.798(2)°,
β=104.869(2)°, and
γ=103.270(2)°,
at a temperature of about 298° K;
(B) a triclinic crystal system and a P-1 space group at a temperature of about 298° K; and
(C) an x-ray powder diffraction pattern with peaks at 20.5 and 25.0° 2θ±0.2° 2θ.

22. Crystalline N,N-dimethyltryptamine (DMT) tartrate, wherein the DMT tartrate forms a unit cell in which there is a ratio of DMT:tartrate of about 1:3 to 1:1.5.

23. The DMT tartrate of claim 22, wherein the ratio of DMT:tartrate is about 1:2.

24. Crystalline N,N-dimethyltryptamine (DMT) tartrate, wherein the DMT tartrate is characterized by one or both of one of:
(A) unit cell dimensions of
a=7.57270(10) Å,
b=9.52180(10) Å,
c=23.7834(4) Å,
α=90°,
β=90.6530(10)°, and
γ=90°,
at a temperature of about 297° K; and
(B) a monoclinic crystal system and a P2₁/n space group at a temperature of about 298° K.

25. The crystalline N,N-dimethyltryptamine (DMT) tartrate of claim 24, wherein the DMT tartrate is characterized by both of:
(A) unit cell dimensions of
a=7.57270(10) Å,
b=9.52180(10) Å,
c=23.7834(4) Å,
α=90°,
β=90.6530 (10)°, and
γ=90°,
at a temperature of about 297° K; and
(B) a monoclinic crystal system and a P2₁/n space group at a temperature of about 298° K.

26. The crystalline N,N-dimethyltryptamine (DMT) fumarate of claim 20, wherein the DMT fumarate is characterized by both of:
(A) unit cell dimensions of
a=7.7447(3) Å,
b=9.3258(4) Å,
c=12.4691(4) Å,
α=102.798(2)°,
β=104.869(2)°, and
γ=103.270(2)°,
at a temperature of about 298° K; and
(B) a triclinic crystal system and a P-1 space group at a temperature of about 298° K.

27. The crystalline N,N-dimethyltryptamine (DMT) fumarate of claim 20, wherein the DMT fumarate is characterized by both of:
(A) unit cell dimensions of
a=7.7447(3) Å,
b=9.3258(4) Å,
c=12.4691(4) Å,
α=102.798(2)°,
β=104.869(2)°, and
γ=103.270(2)°,
at a temperature of about 298° K; and
(B) an x-ray powder diffraction pattern with peaks at 20.5 and 25.0° 2θ±0.2° 2θ.

28. The crystalline N,N-dimethyltryptamine (DMT) fumarate of claim 20, wherein the DMT fumarate is characterized by both of:
(A) a triclinic crystal system and a P-1 space group at a temperature of about 298° K; and
(B) an x-ray powder diffraction pattern with peaks at 20.5 and 25.0° 2θ±0.2° 2θ.
